(19) United States
(12) Patent Application Publication
(10) Pub. No.: US 2017/0072150 A1
(43) Pub. Date: Mar. 16, 2017

(54) Halotherapy Devices and Methods

(71) Applicant: Wake Forest University Health Sciences, Winston-Salem, NC (US)

(72) Inventors: Charles Lankau, Winston-Salem, NC (US); Daniel Porada, Winston-Salem, NC (US); Brandon Hall, Wilmington, NC (US)

(21) Appl. No.: 15/266,482

(22) Filed: Sep. 15, 2016

Related U.S. Application Data
(60) Provisional application No. 62/218,928, filed on Sep. 15, 2015.

(51) Int. Cl. A61M 15/00; A61M 15/08

(52) U.S. Cl. CPC A61M 15/0085; A61M 15/0021; A61M 15/009; A61M 15/08; A61M 2205/75; A61M 2207/00; A61M 2202/06; A61M 2205/8206

(57) ABSTRACT

Devices and methods for administering halotherapy are provided for treating respiratory or skin conditions. Variations include a portable devices as well as stationary devices suitable for administering therapy in permanent settings (e.g., in the home).

### RELATED APPLICATIONS

The present application claims priority to U.S. Provisional Patent Application No. 62/218,928, filed Sep. 15, 2015. The disclosure of U.S. Provisional Patent Application No. 62/218,928 is hereby incorporated by reference in its entirety herein. References cited herein are also incorporated by reference in their entirety.

### FIELD

The apparatus and methods relate to therapeutic uses of airborne salt particulates. Breathing the salt delivers the particulates to the airways and lungs of subjects having respiratory disorders. Exposing the skin to airborne salt may be beneficial for treating some skin conditions.

### BACKGROUND

Inhalation of the airborne saline dust in natural salt mines or caves has long been used as a treatment for respiratory conditions. This so-called speleotherapy relies on the increased salt content of the air in the salt cave microclimate. Halotherapy is derived from speleotherapy and involves administration of breathable salt air similar to that found in the salt cave microclimate. Various approaches to administering the salt air therapy include dry salt pipes, salinizers, or sessions in artificial salt chambers designed to recreate the physical setting of the salt caves as well as the salt air microclimate. In recent years, there has been an increase in the commercial availability of halotherapy as an alternative complementary treatment to supplement traditional medical treatments. As such halotherapy chambers are now found in many locations worldwide. Most traditionally, halotherapy is taken in salt caves or mines, where the presence of enormous amounts of salt naturally increase the concentration of salt in the air.

Salt pipes and salt chambers are both used by sufferers of COPD and other respiratory ailments, such as asthma, to alleviate their symptoms. Although there are salt chambers and salt pipes available, these function simply by inhaling air which has been in the presence of or passively passed across salt crystals.

There are commercial salinizers that utilize ultrasonic frequencies to provide halotherapy in one's home. However, as described in detail below, these devices differ from the halotherapy devices of the present invention both structurally and functionally, in that they require a salt solution, such that the user must repeatedly create their own salt solutions for refills.

Therefore, for subjects with respiratory conditions, what is needed is a device for administering halotherapy that is more convenient and less expensive than visiting salt mines or artificial salt chambers, more effective than dry salt pipes, and easier to manage than a salinizer.

### SUMMARY

The invention described herein comprises an apparatus or device for administering halotherapy, including at least a salt-loaded filter. Some embodiments of the device further include a component that generates vapor or steam from water, a power source, a water reservoir, and a salt-loaded filter.

In some embodiments, the component that generates vapor or steam is a component that generates ultrasonic frequencies. In some embodiments, a component that generates ultrasonic frequencies is an ultrasonic transducer. In some embodiments, an ultrasonic transducer comprises a piezoelectric crystal.

In some embodiments, vapor or steam generated by the apparatus or device is forced to pass through a salt-loaded filter before escaping the apparatus. In some embodiments, the component that generates vapor or steam is connected to the power source. In some embodiments, the component that generates vapor or steam is positioned to utilize water from the water reservoir.

Some embodiments include a water reservoir which may have a capacity of at least about 2.5 liters. In other embodiments, a water reservoir may have a capacity of less than about 0.4 liters. In still other embodiments, the water reservoir may have a capacity of about 0.4-2.5 liters.

Some embodiments of an apparatus or device according to the invention may further include a mouthpiece and/or a nasal cannula, either of which is connected to an opening where vapor or steam escapes the apparatus. Some embodiments further comprise a mesh screen, an O-ring gasket, or a fan.

In some aspects, the invention described herein includes a method for loading a 3-dimensional filter with salt, including: providing a super-saturated salt solution, submerging the 3-dimensional filter in the super-saturated salt solution for a period of time, and drying the 3-dimensional filter. In some embodiments, the drying is performed in a convection oven.

In some methods for loading a salt filter, essential oils, flavors, or scents are incorporated into the super-saturated salt solution or otherwise added to the filter.

Embodiments of the invention described herein also include methods for administering halotherapy, including providing an enclosed space that contains both a device that includes at least a salt-loaded filter and a subject to receive the halotherapy.

### BRIEF DESCRIPTION OF FIGURES

FIG. 1 illustrates a prototype adapted from a humidifier, according to one embodiment of the invention.

FIG. 2 shows views of a prototype filter for use with a stationary apparatus, according to one embodiment of the invention.

FIG. 3 is a photograph of the humidifier modified to embody the invention, as illustrated in FIG. 1.

FIG. 4 is a photograph of the fully assembled modified apparatus illustrated in FIG. 1.

FIG. 5 shows three views of a portable halotherapy device, according to one embodiment of the invention.

FIG. 6 shows detail of the salt filter for the portable device shown in FIG. 5, according to one embodiment of the invention.

FIG. 7 shows an exploded view of the portable halotherapy device, according to one embodiment of the invention.

### DETAILED DESCRIPTION

Provided herein are devices and methods for administering halotherapy. Halotherapy provides breathable microscopic salt particles for subjects with respiratory disorders or conditions. Unlike devices currently available, the devices of the present invention actively vaporize or aerosolize salt for breathing. Also unlike devices currently available, the devices of the present invention utilize either steam or water vapor as a method for carrying the salt into the respiratory tissues. Devices of the present invention can benefit sufferers of COPD and other respiratory ailments, such as asthma, to alleviate their symptoms.

Also unlike devices currently available, the devices of the present invention do not require the user to prepare salt solutions. Nor do they require frequent cleaning or a special cleaning solution, like a salinizer (due to salt and mineral deposits on the ultrasonic cell and inside the water tank). The devices of the present invention thus provide a more convenient halotherapy system, because the user or subject does less preparation and cleaning.

As used herein, the term “subject” refers to any mammal, in particular a human, at any stage of life.

“About” and “around,” as used herein to modify a numerical value, indicate a defined range around that value. If “X” were the value, “about X” or “around X” would generally indicate a value from 0.95X to 1.05X including, for example, from 0.98X to 1.02X or from 0.99X to 1.01X. Any reference to “about X” or “around X” specifically indicates at least the values X, 0.95X, 0.96X, 0.97X, 0.98X, 0.99X, 1.01X, 1.02X, 1.03X, 1.04X, and 1.05X. Thus, “about X” and “around X” are intended to teach and provide written description support for a claim limitation of, e.g., “0.98X.”

Embodiments of a halotherapy device comprise at least a salt-loaded filter. A salt-loaded filter can be coated or crystallized with any salt desirable for halotherapy. The type of salt may depend on prescription or user preference. For example, a salt-loaded filter may include Himalayan pink salt, Polish mine salt, Dead Sea salt, epsom salts, or other desirable halotherapy salts. Combinations of salts may also be desirable.

A filter having extensive surface areas for holding salt can be salt-loaded by submerging the filter into a super-saturated salt solution for a period of time (e.g., about 15 minutes). A salt-loaded filter can then be dried in a convection oven. Variations on this method of preparing a salt-loaded filter are possible, such as submerging the filter for shorter or longer periods of time or different methods of drying the loaded filter. Other methods for encouraging crystallization may be employed as described in the art. Available surface area can be increased by additional filter features. These extensive surface areas also facilitate dispersion of salt particulates into the vapor as an aerosol during the halotherapy administration.

In some embodiments, surfaces on the filter may be modified to encourage better loading of salt crystals. For example, the filter surfaces can be dimpled or mottled to increase available surface area for salt crystallization. The filter may comprise a porous material. The surfaces of the filter can also be treated with any agent that encourages more deposition of salt or deposition in a manner that may be more efficiently vaporized by cool vapor or steam.

In particular environments, where the air contains high levels of humidity (either naturally or artificially), a salt-loaded filter can be placed in the environment and a user can be positioned in proximity to the filter. For example, a salt-loaded filter can be placed in a steam room, and a person who sits next to the filter can benefit from therapeutic properties of the salt that is aerosolized by the effects of the steam on the salt-loaded filter.

Some embodiments of halotherapy devices include a mechanism for generating steam or vapor to release and aerosolize salt from the salt-loaded filter. When activated, a halotherapy device can generate vapor or steam from distilled water contained within an onboard, refillable water reservoir. The vapor or steam can then be passed across a filter cartridge carrying charges of salt crystals, such that salt particulates are carried with the steam or vapor into the air. Some embodiments (especially a larger apparatus, e.g.) may include a small fan to facilitate dispersion of the vapor from the chamber in which it is formed through a salt filter and into the ambient air.

In certain embodiments, the halotherapy devices of the present invention include a water reservoir. The size of the reservoir may depend on the intended use of the particular device. For example, stationary devices may accommodate much larger water reservoirs than portable devices. In some embodiments, the water reservoir for a stationary apparatus may have a capacity of at least about 2.5 liters (0.66 gallons).

In some embodiments, an apparatus or device of the invention may generate vapor or steam from the water. The water may be distilled water. In some embodiments, the generation of vapor or steam causes the vapor or steam to pass through or across a salt-loaded filter. In some embodiments the vapor or steam picks up tiny salt particulates from the surfaces of the salt-loaded filter and disperses the particulates into the air along with the vapor or steam. In some embodiments, one or more O-rings around the filter provides a seal between the side of the filter and the side of the air column, such that vapor cannot escape the apparatus without first moving through the salt-loaded filter. Or other means of sealing any spaces surrounding the filter may be employed, so as to force as much vapor as possible through the filter.

In some embodiments, heating the water generates steam. For example, a heating coil may be wrapped around the water reservoir to heat the water. Or other mechanisms for heating water may be included.

In some embodiments, an apparatus of the present invention may utilize ultrasonic frequencies to vaporize distilled water, for example via a piezoelectric crystal positioned within the water reservoir. Thus when the apparatus is powered on, the electricity applied to the piezoelectric crystal causes vibrations in the crystal that generate ultrasonic frequencies. The application of ultrasonic frequencies to water can create a vapor (i.e., microscopic water droplets).

The microscopic water droplets in the water vapor produced by ultrasonic frequencies may then be passed across a filter material carrying charges loaded with one or more of a variety of salt crystals in appropriate concentrations to reach therapeutic levels when inhaled by the user as either a steam or a warm or cold vapor.

The use of piezoelectric material to generate vapor may be advantageous in several ways. The vapor is generated almost instantly when the device is turned on, as compared to heating water to generate steam, which requires waiting some period of time for the water to heat up before the steam will start to be produced. Also a piezoelectric crystal generally uses less energy/electricity than a heating element. Piezoelectric ultrasonic transducers are commercially available components. An ultrasonic transducer (which contains a piezoelectric crystal) may be positioned below a water reservoir and maintain contact with a membrane that comprises a portion of the bottom of the water reservoir. When an ultrasonic transducer is powered on (e.g., via Li-ion battery or other means), it vibrates the membrane at a frequency that produces water vapor. Modulation of the voltage can alter the intensity of water vapor production.

Some embodiments of a halotherapy apparatus may include a power source, such as a battery. Portable embodiments especially may utilize a battery, such as a lithium ion rechargeable battery similar to those typically used in electronic cigarettes. Or other batteries or alternative power sources may be used. Some embodiments may include a potentiometer for controlling voltage. A stationary apparatus may simply be plugged in using a typical cord, plug, and receptacle, for example.

In some embodiments, the halotherapy devices may generate air having salt content in ranges of about 0.1-1.0, 1.0-3.0, 2.0-3.0, 3.0-5.0, 7.0-9.0, 0.5-9.0, 0.1-10.0, 0.1-12.5, 0.1-15.0 mg/m³, or even higher concentrations of salt particles in the vapor/steam for some conditions (e.g., for treating skin conditions). Different respiratory conditions may necessitate different levels of salt content in the halotherapy air, as described in Chervinskaya and Zilber (1995)J. Aerosol Med. 8:221-232 (See, e.g., Table 3).

In some embodiments, the vaporized salt crystals will be a medically acceptable size of about 3-5 μm (micrometers) on average. In some embodiments, the size of the vaporized salt crystals may average about 0.5-14.0, 0.5-12.0, 0.5-10.0, 0.5-9.0, 0.5-8.0, 0.5-7.0, 1.0-7.0, or 2.0-10.0 μm. In some embodiments, the salt crystals may occur in a wide range of sizes. Halotherapy devices may be adjustable to generate more of a given particle size, based on user preferences or needs. For example, larger particles may be desirable to target higher airways, while the smallest particles may be desirable to reach the smallest, most distal airways and alveoli.

A halotherapy apparatus may include mechanisms for adjusting parameters such as the rate of vapor or steam being generated, average size of the particulate salts in the vapor or steam, heat, timing, or other features. These may be adjusted to accommodate user preference or a prescription from a healthcare provider.

The vaporized salt along with the warm/cool steam may be inhaled and delivered to the lungs by positioning a subject (e.g., human or animal) near a stationary halotherapy device, such that they breathe the salt air emissions. An enclosed space, such as a room or chamber, can facilitate administration of the halotherapy by simultaneous placement of a halotherapy device in an enclosed space with a subject who receives the therapy.

In some stationary apparatus embodiments, the device may have the capacity to fill a small- or medium-sized room with salt vapor (e.g., about 100-250 square feet). Some embodiments are larger devices with more capacity. For example, the size of the water reservoir, the size of the filter, and/or the amount of vapor produced per unit time may be increased for stationary devices. In some embodiments, a stationary halotherapy device may optionally include a removable mouthpiece and/or nasal cannula or other similar extension for direct delivery to the respiratory passages of an individual user.

The combination of salt particulate therapy and the therapeutic effects of breathing steam may provide an optimal non-drug based therapy for a number of respiratory conditions with unprecedented efficacy.

In certain embodiments, the halotherapy device of the invention is portable. Embodiments of a portable halotherapy device provide a convenient therapeutic alternative to the other options for halotherapies which are being used widely to treat or alleviate the symptoms of a variety of respiratory diseases or complications. In some embodiments, the steam or vapor generated by the portable device may alternatively be inhaled directly from the device via a mouthpiece, or from an extension of the device (e.g., a nasal cannula). A mouthpiece may be removable, washable, and/or disposable

Embodiments of a portable halotherapy device may generally include smaller features, such as a smaller filter and smaller water reservoir. For example, the water reservoir may have a capacity less than 0.4 liters or 0.1 gallons. Or the water reservoir may be smaller, such as about 0.025, 0.05, 0.075, 0.10, 0.15, 0.20, 0.25, 0.30, or 0.35 liters.

Embodiments of a stationary halotherapy apparatus may generally include larger features, such as a larger filter that has capacity to hold more salt. A stationary device may also include a larger reservoir for water. For example, the water reservoir may have a capacity of at least about 2.5 liters or 0.66 gallons. Or the water reservoir may be larger, such as at least about 2.75, 3.0, 3.25, 3.5, 3.75, 4.0, 4.25, 4.5, or 5.0 liters capacity.

Additional optional structural features may include: a fine mesh layer in the air column (i.e., where the vapor or steam moves from the water reservoir to the filter) to prevent water from splashing onto the filter; one or more O-ring gaskets surrounding the filter where it meets the walls of the air column in the apparatus, to prevent vapor from leaking out of the apparatus around the sides of the filter; and (especially in larger devices, e.g.) a small fan near the base to facilitate movement of vapor or steam through the filter and into the ambient air.

The structure of halotherapy devices may comprise a variety of materials. Hard materials such as plastics, metals, fiberglass, etc. may be appropriate for some embodiments or components. The components may be made with injection molding, 3-D printing, or any of the other methods known in the art. In some embodiments texture may be added to flat surfaces of the devices, such as interior or exterior surfaces. Texture added to exterior surfaces may facilitate gripping the devices. Textures added to interior surfaces may facilitate crystallization of salt in the process of loading a filter with salt (or the vaporization of the salt from the filter by the vapor or steam generated).

The salt-loaded filters can be replaceable in some embodiments. For example, refill filters may be available for purchase by consumers. In some embodiments, an indicator may alert the user that a filter is almost emptied of salt and needs replacement. The indicator may utilize an electric current to determine the salt content remaining in the filter, or other indicators may be employed. In some embodiments, a salt-loaded filter can be re-loadable after use. The choice of materials for a salt-loaded filter can depend on whether disposable use or long-term use is desirable.

Some versions of halotherapy devices may include additional features, such as essential oils incorporated into the salt-loaded filter for specific scents or flavors. In such embodiments, particular combinations of specific salts with specific essential oils may offer specific benefits for particular respiratory conditions. For example, eucalyptus and peppermint reportedly offer benefits for respiratory conditions. Scents or flavors may also be incorporated into a mouthpiece or other components near the point where the vapor escapes the apparatus, instead of the filter. Or they may be added to the water in the reservoir. Devices may also include handles. Some embodiments may include a charging stand, for example if a rechargeable battery is part of the device.

The illustrative examples provided with this disclosure are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative examples but, like the illustrative examples, should not be used to limit the present disclosure.

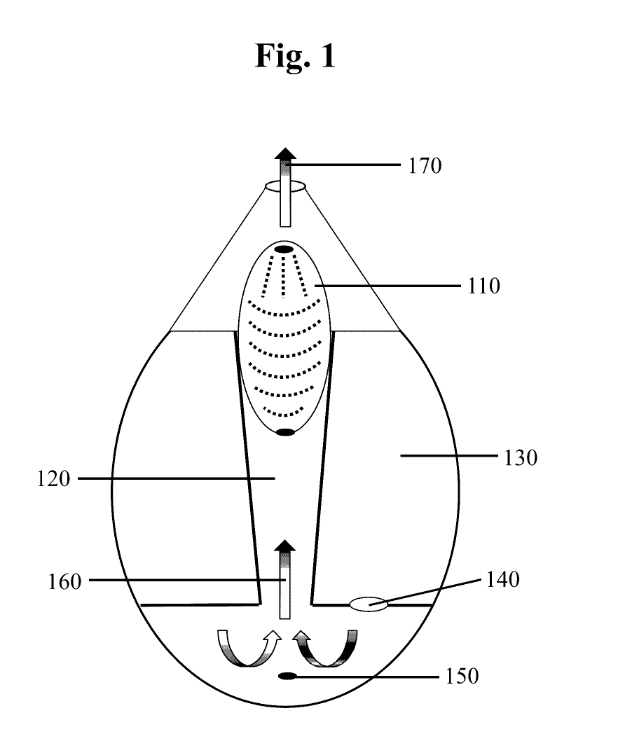

FIG. 1 illustrates a prototype adapted from a humidifier type device, according to one embodiment of the invention. A porous salt-loaded filter 110 was fitted into the top of an air column 120 of a humidifier type apparatus. A water reservoir 130 allows water to move through an aperture 140 into the base of the apparatus where a piezoelectric crystal 150 generates ultrasonic frequencies to create water vapor 160. The water vapor 160 rises through the air column 120 and enters the salt-loaded filter 110 before escaping the apparatus as a salt vapor 170. Forcing the water vapor 160 through the pores in the filter 110 causes the vapor to carry salt particulates out of the device and into the ambient air.

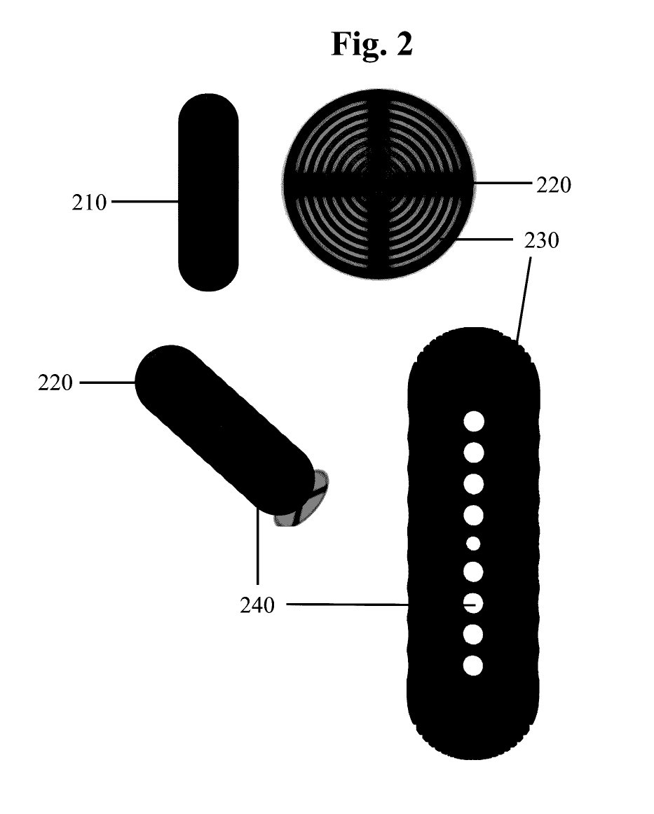

FIG. 2 shows different views of a prototype filter for use with a larger apparatus, according to one embodiment of the invention. This filter was 3-D printed and incorporated into a humidifier type device, as shown in FIG. 1, to prototype a stationary apparatus embodiment of the invention. The custom filter was loaded with Himalayan salt by submerging the filter into a super-saturated salt solution for 15 minutes and then drying the filter in a convection oven. The salt-loaded filter was then fitted into the top section of the central air column in the modification of a humidifier type apparatus, as shown in FIG. 1. A side view is shown as 210. The top and bottom parts of the filter 220 are similar or identical and have numerous slits 230 designed for vapor in-flow and out-flow. The sides of the filter have additional holes 240, and generally the filter has maximized inside surface area for salt deposition and ionization. A cross-section of the inside of this filter is shown at right in FIG. 2. The cross-section illustrates the continuation of concentric circles (broken by solid segments for structural integrity) of the top and bottom pieces 220, through the filter 230. This configuration is only one example, and other filter designs may maximize and optimize available surface areas for salt deposition and/or salt vaporization.

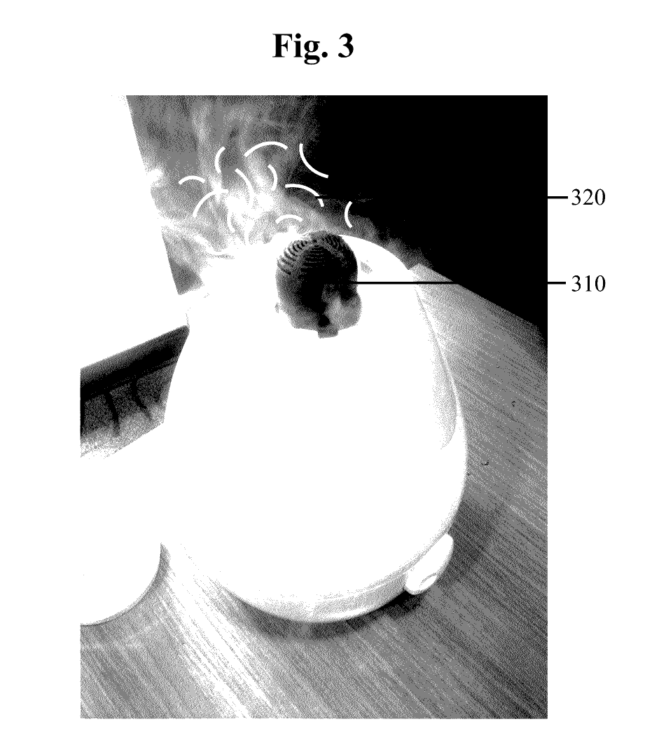

FIG. 3 is a graphically enhanced photograph of an embodiment of the invention, as illustrated in FIG. 1, but without the top piece (such that the filter is visible). The salt-loaded filter 310 illustrated in FIG. 2 is fitted into the top of the air column, such that rising water vapor is forced through the salt-loaded filter 310 before escaping the apparatus into the ambient air. Thus when the apparatus is powered on (as here), the device generates salt-carrying vapor (graphically enhanced as 320) which is visible around the top of the filter 310.

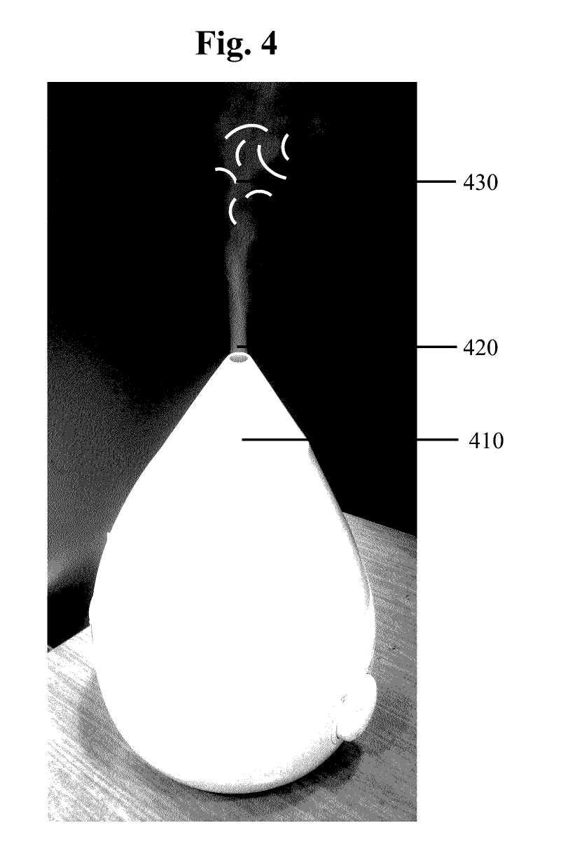

FIG. 4 is a graphically enhanced photograph of the fully assembled modified apparatus illustrated in FIG. 1. The lid 410 is added (as compared to FIG. 3 without the lid), such that the salt-carrying vapor is forced through the small aperture at the top of the apparatus 420 into the ambient air. The apparatus is powered on and is therefore visibly generating salt-carrying vapor 430.

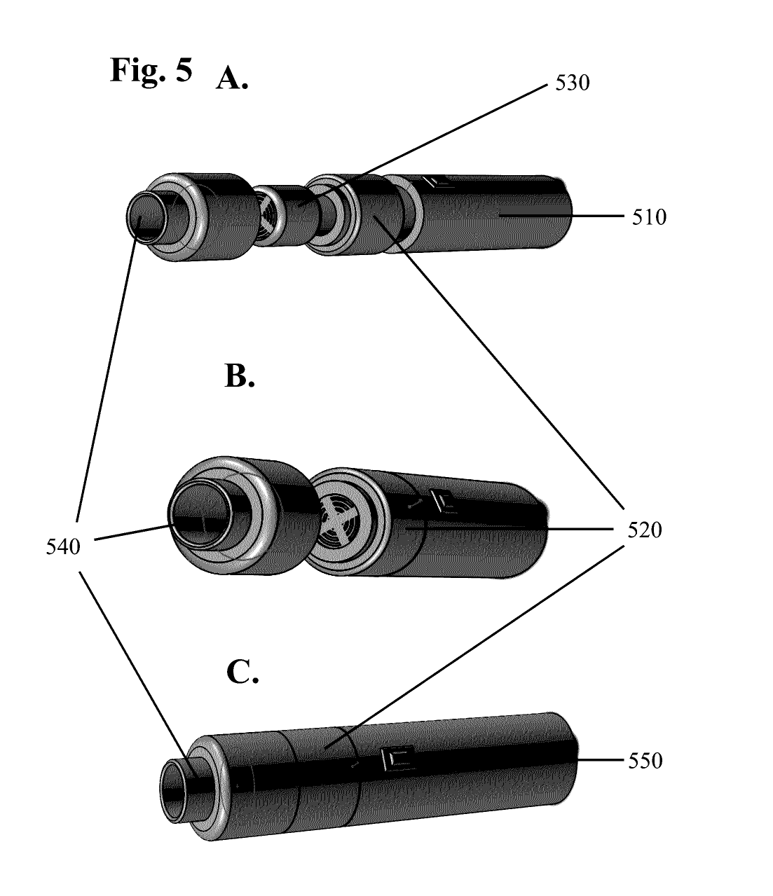

FIG. 5 shows three views of a portable halotherapy device according to one embodiment of the invention. Panel A is an exploded view, showing four separate pieces: The base 510, the salt chamber 520, the salt-loaded filter or cartridge 530, and the mouthpiece 540. Panel B shows the salt chamber 520 housing the salt-loaded filter or cartridge 530, and attached to the base piece, but without the mouthpiece 540 attached. Panel C shows the fully assembled portable halotherapy device, with a button 550 on the base piece for powering on the device, thereby activating the battery. The base 510 houses the water reservoir, battery, and piezoelectric crystal.

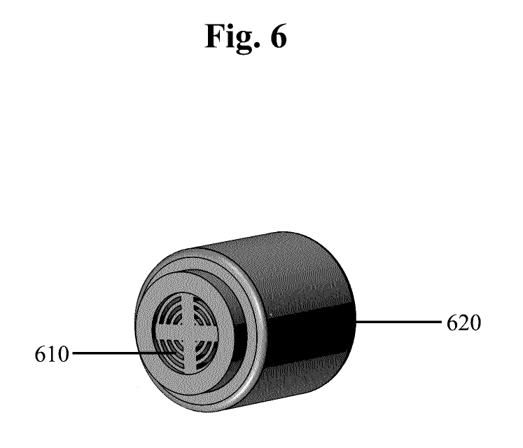

FIG. 6 shows detail of an embodiment of a salt filter or cartridge for a portable device. As explained above, the inside of a filter should maximize surface areas for deposition of salt in the filter-loading process. As with the larger filter shown in FIG. 2, the top and bottom parts of the filter are similar and have numerous slits 610 designed for vapor in-flow and out-flow. However, in this embodiment, the sides of the filter 620 do not have additional holes, as they are not exposed to air flow.

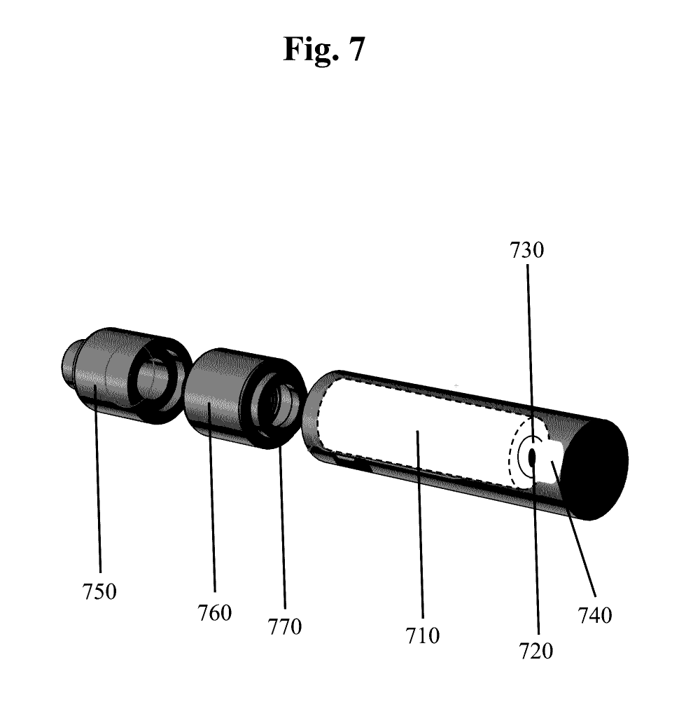

FIG. 7 shows an exploded view of a portable halotherapy device, according to one embodiment of the invention. This view illustrates the water reservoir 710, an ultrasonic transducer 720 comprising a piezoelectric crystal in contact with a membrane 730 at the bottom of the water reservoir 710, and a rechargeable Li-ion battery 740 inside the base section of the device. Also visible are the mouthpiece 750, the salt chamber 760, and coupling mechanisms between segments 770. In the embodiment shown in FIG. 7, the ultrasonic transducer 720 (which contains a piezoelectric crystal) sits below the water reservoir 710 and maintains contact with the membrane 730 that comprises a portion of the bottom of the water reservoir 710. When the ultrasonic transducer 720 is powered on via the Li-ion (lithium ion) battery 740, it vibrates the membrane 730 at a frequency that produces water vapor.

Embodiments of the disclosed halotherapy devices may render the alleviation of symptoms much easier and simpler for a subject, as it may be more convenient and/or less expensive than the conventional salt chamber therapy sessions. The portable version is also handheld and rechargeable for optimal convenience. It may be used, like a salt pipe, whenever symptoms begin to present, and may thus potentially reduce the need for expensive therapeutic drugs and inhalers, and meanwhile may also reduce or eliminate the adverse health effects often reported for chronic users of asthma inhalers.

Embodiments of the disclosed halotherapy devices may address a wide variety of respiratory difficulties and conditions, including but not limited to chronic obstructive pulmonary disease (COPD), cystic fibrosis, asthma, allergies, difficulties caused by smoking, the common cold, chest infections, and bronchitis. Due to their simple design and functionality, the devices disclosed herein are well-suited to alleviating the symptoms of any number of respiratory conditions. Some halotherapy reportedly also offers benefits for certain skin conditions, and the invention described herein may be applied for that purpose by simply exposing affected skin to the halotherapy steam or vapor.

The foregoing description of certain examples, including illustrated examples, as well as the Examples described below, are presented only for the purpose of illustration and description and are not intended to be exhaustive or to limit the disclosure to the precise forms disclosed. Numerous modifications, adaptations, and uses thereof will be apparent to those skilled in the art without departing from the scope of the disclosure.

### EXAMPLES

### Example 1

### A Stationary Halotherapy Apparatus

An ultrasonic humidifier type apparatus was modified to prototype an embodiment of the invention. An ultrasonic cool mist humidifier in full size (not miniature), appropriately sized for a stationary apparatus, was adapted to embody the invention disclosed herein. The apparatus has a removable water tank that holds 0.9 gallons. Its dimensions are 9-¾″ diameter and 14-¼″ tall. The unmodified apparatus is suitable for humidifying a room measuring approximately 250 square feet.

The modified apparatus is shown in FIGS. 1-4. To modify the humidifier type apparatus, a custom filter with many inner surfaces holes or pores (shown in FIG. 2) was created with a 3-D printer. The custom filter was loaded with Himalayan salt by submerging the filter into a super-saturated salt solution for 15 minutes and then drying the filter in a convection oven. The salt-loaded filter was then fitted into the top section of the central air column in the Ultrasonic Cool Mist Humidifier, as shown in FIG. 1. Two large O-rings around the filter provided a seal between the side of the filter and the side of the air column, such that vapor could not escape the apparatus without first moving through the salt-loaded filter.

As illustrated in FIG. 1, when the apparatus is powered on, the piezoelectric crystal near the base of the apparatus generates vapor from distilled water in the reservoir. The vapor rises through an air column in the center of the apparatus and is forced through the many holes in the salt-loaded filter, in order to escape the apparatus.

Several optional features are not visible in the figures. First, a fine mesh layer was added to the air column below the salt-loaded filter but well above the surface of the water to prevent water from splashing onto the filter. Second, O-ring gaskets were fitted to surround the filter where it meets the walls of the air column in the apparatus, to prevent vapor from leaking out of the apparatus around the sides of the filter. And third, the unmodified humidifier apparatus includes a small fan near the base, which was not removed in the prototype. The fan facilitates dispersion of vapor through the filter, out of the apparatus, and into the ambient air.

Experiments with this modified apparatus demonstrated salt crystals deposited onto both construction paper and sponges by the vapor. Microscopic inspection of the deposited salt indicated particulate sizes ranging from 1-14 μm approximate diameter. Laboratory analysis of water vapor produced via ultrasonic frequencies also indicates a drop size of approximately 1-14 μm. As the salt crystals are carried by the droplets, they must be less than 14 μm in diameter.

### Example 2

### A Portable Halotherapy Device

A halotherapy device can be hand-held and portable, such that it is easily carried away from home and used in the car or in other locations. A portable device was modeled in SOLIDWORKS®, as illustrated in FIGS. 5-7, and has the following dimensions:
Base Diameter: 40 mm
Center Height: 206.02 mm
Mouthpiece Inner Diameter: 20 mm
Mouthpiece Outer Diameter: 24 mm
Water tank height: 73 mm
Water tank inner diameter: 30 mm
Water tank volume: approx. 51.6 mL

This portable device is somewhat larger than a typical electronic cigarette but still much smaller than the stationary apparatus described in Example 1. When powered on, the ultrasonic frequencies generated by the piezoelectric crystal create water vapor which moves through the salt-loaded filter section and into the mouthpiece. The user places the mouthpiece at her lips and inhales the salt air directly into the respiratory airways.

Like the stationary apparatus example, the portable device utilizes a replaceable salt-loaded filter or cartridge which may be purchased already loaded with particular salts. The water reservoir volume of approximately 51.6 mL corresponds to a capacity for saturating approximately 5.4 kg dry air to 60% humidity.

### Example 3

### A Salt-Loaded Filter Halotherapy Device

A halotherapy device can include a salt-loaded filter placed in a steamy environment. In one example, the filter is a 3-D lattice structure with extensive surface area. The filter is loaded with salt by submersion in a super-saturated Himalayan pink salt solution for three 15-minute periods of time, with one hour of drying time after each loading period.

The salt-loaded filter is placed in a steam room or sauna, and a person sits in the sauna such that her face is positioned about 20 cm from the filter. The salt is aerosolized by the steam in the environment, and the person breathes in the aerosolized salt for therapy.

1. An apparatus or device for administering halotherapy comprising a salt-loaded filter.

2. The apparatus of claim 1, further comprising a component that generates vapor or steam from water, a power source, and a water reservoir.

3. The apparatus of claim 2, wherein the component that generates vapor or steam is a component that generates ultrasonic frequencies.

4. The apparatus of claim 2, wherein the vapor or steam is forced to pass through the salt-loaded filter before escaping the apparatus.

5. The apparatus of claim 3, wherein the component that generates ultrasonic frequencies is an ultrasonic transducer.

6. The apparatus of claim 5, wherein the ultrasonic transducer comprises a piezoelectric crystal.

7. The apparatus of claim 2, wherein the component that generates vapor or steam is connected to the power source.

8. The apparatus of claim 2, wherein the component that generates vapor or steam is positioned to utilize water from the water reservoir.

9. The apparatus of claim 2, wherein the water reservoir has a capacity of at least 2.5 liters.

10. The apparatus of claim 2, wherein the water reservoir has a capacity of less than 0.4 liters.

11. The apparatus of claim 2, wherein the water reservoir has a capacity of 0.4-2.5 liters.

12. The apparatus of claim 2, further comprising a mouthpiece connected to an opening where vapor or steam escapes the apparatus.

13. The apparatus of claim 2, further comprising a nasal cannula connected to an opening where vapor or steam escapes the apparatus.

14. The apparatus of claim 2, further comprising a mesh screen.

15. The apparatus of claim 2, further comprising an O-ring gasket.

16. The apparatus of claim 2, further comprising a fan.

17. A method for loading a 3-dimensional filter with salt, comprising:
providing a super-saturated salt solution,
submerging the 3-dimensional filter in the super-saturated salt solution for a period of time, and
drying the 3-dimensional filter.

18. The method of claim 17, wherein the drying is performed in a convection oven.

19. The method of claim 17, wherein essential oils, flavors, or scents are incorporated into the super-saturated salt solution or otherwise added to the filter.

20. A method for administering halotherapy, comprising providing an enclosed space comprising:
a device comprising at least a salt-loaded filter, and
a subject to receive the halotherapy.
